(19) United States
(12) Patent Application Publication
(10) Pub. No.: US 2008/0242977 A1
(43) Pub. Date: Oct. 2, 2008

(54) SYSTEMS, METHODS AND APPARATUS FOR LONGITUDINAL/TEMPORAL ANALYSIS OF PLAQUE LESIONS

(75) Inventors: Saad Ahmed Sirohey, Pewaukee, WI (US); Sandeep Dutta, Waukesha, WI (US); Gopal B. Avinash, New Berlin, WI (US)

Correspondence Address: RAMIREZ & SMITH, PO BOX 341179, AUSTIN, TX, 78734, US

(73) Assignee: GENERAL ELECTRIC COMPANY, Schenectady, NY (US)

(21) Appl. No.: 11/694,911

(22) Filed: Mar. 30, 2007

(51) Int. Cl. A61B 5/05

(52) U.S. Cl. 600/425

(57) ABSTRACT

Systems, methods and apparatus are provided through which in some embodiments detection of a change in characteristics of plaque in a longitudinal exam is automated for the purpose of assessing change in disease due to therapy, patient behavior modifications or follow-up. In some embodiments, diagnosis and treatment of arterial lesions includes obtaining a plurality of sets of computed-tomography images of at least one arterial plaque lesion, wherein each set of computed-tomography images are acquired at a different time, then storing the computed-tomography images in a database and analyzing arterial plaque variations in the sets of computed-tomography images for changes in at least one parameter.

### FIELD OF THE INVENTION

This invention relates generally to medical imaging, and more particularly to graphical image analysis of lesions in medical images.

### BACKGROUND OF THE INVENTION

Cardiovascular related deaths number more than 500,000 annually in the USA, and much more globally. A major portion of the cardiovascular deaths are attributed to coronary artery disease, where the chief culprit is the build up of plaque, specifically soft-plaque and its ruptures. Typically in X-ray or non-contrasted computed-tomography (CT) medical imaging the soft-plaque is not easily detectable. Calcified plaque on the other hand has been used as a surrogate for the presence of soft plaque, with the reasoning being that calcified plaque is a by-product of ruptured soft plaque.

Coronary plaque is classified into six stages according to the Stary scale. The Stary scale classifies atherosclerotic lesions. According to general consensus, determining the presence of plaque in stages 4 and 5 of the Starry scale is critical as stages 4 and 5 constitute critical vulnerable plaque that could lead to rupture or dislodging of the plaque causing blockages leading to myocardial infarction (MCI). The most prominent standard for determining plaque and constituency of the plaque is intravascular ultrasound (IVUS), however IVUS is only performed on symptomatic patients due to the invasive nature of IVUS. Patients who are symptomatic of MCI are already at an advanced stage and past non-invasive therapy options.

With the advent of cardiac volume computed tomography (VCT) and the ever increasing spatial and temporal resolution of VCT and the impending arrival of high definition (HD) VCT, imaging a contrasted study of the heat gated to mitigate heart motion is feasible. From these images, distinguishing plaque from lumen and from calcification is now within reach.

Plaque deposits (e.g. soft plaque, hard plaque and mixed plaque) in the coronary and carotid vessels of patient's changes over time due to a number of clinical factors. Moreover, available drugs can be administered to a heart patient that can cause significant changes in the composition of dangerous soft and mixed plaque deposits to a composition of benign calcified plaque lesions. Plaque deposits can also break free and move to very dangerous narrower regions of a vessel. However, many of these important and significant changes are not necessarily noticed by health care providers. For the reasons stated above, and for other reasons stated below which will become apparent to those skilled in the art upon reading and understanding the present specification, there is a need in the art to track changes in coronary arterial plaque lesions over time.

### BRIEF DESCRIPTION OF THE INVENTION

The above-mentioned shortcomings, disadvantages and problems are addressed herein, which will be understood by reading and studying the following specification.

In one aspect, detection of a change in characteristics of plaque in a longitudinal exam is automated for the purpose of assessing change in disease due to therapy, patient behavior modifications or follow-up.

In another aspect, diagnosis and treatment of arterial lesions includes accessing a plurality of images of a patient that were acquired longitudinally and analyzing arterial plaque variations in the plurality of images for changes in which the changes include at least one change in size, at least one change in composition, at least one change in characteristics and at least one change in location. Changes in shape, size, location and composition of plaque lesions show the temporal changes in the disease conditions of a patient.

In yet another aspect, diagnosis and treatment of arterial lesions includes accessing a plurality of sets of computed-tomography images of at least one arterial plaque lesion, wherein each set of computed-tomography images are acquired at a different time, storing the computed-tomography images in a database and analyzing arterial plaque variations in the sets of computed-tomography images for changes in at least one parameter.

In still another aspect, a volumetric computer assisted reading (VCAR) system includes a software means operative on a processor to detect changes in lesions based on a plurality of studies at a plurality of times and to generate a graphical color coded representation of the changes in each lesion with the plurality of times. Historical measurements provide a user friendly graphical way for the healthcare practitioners to see the temporal effects of the clinical treatments and the progression/regression of the lesions.

Systems, clients, servers, methods, and computer-readable media of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a block diagram of an overview of the system to support diagnosis and treatment of arterial lesions, according to an embodiment;

FIG. 2 is a flowchart of a method to support diagnosis and treatment of arterial lesions according to an embodiment;

FIG. 3 is a flowchart of a method of analyzing the plurality of images for changes in arterial plaque, according to an embodiment;

FIG. 4 is a flowchart of a method of analyzing the plurality of images for changes in arterial plaque, according to an embodiment;

FIG. 5 is a flowchart of a method of analyzing the plurality of images for changes in arterial plaque, according to an embodiment;

FIG. 6 is a flowchart of a method to support diagnosis and treatment of arterial lesions, according to an embodiment of longitudinal image acquisition;

FIG. 7 is a flowchart of a method to support diagnosis and treatment of arterial lesions, according to an embodiment of longitudinal image acquisition;

FIG. 8 is a flowchart of a method to support diagnosis and treatment of arterial lesions, according to an embodiment that includes computed tomography image acquisition;

FIG. 9 is a flowchart of a method to support diagnosis and treatment of arterial lesions, according to an embodiment that includes magnetic resonance image acquisition;

FIG. 10 is a flowchart of a method to support diagnosis and treatment art to a lesions, according to embodiment that includes computed tomography image acquisition;

FIG. 11 is a flowchart of a method to support diagnosis and treatment of arterial lesions according to an embodiment;

FIG. 12 is a flowchart of a method support diagnosis and treatment of material lesions, according to the climate that provides a visual cue of changes;

FIG. 13 is a flowchart of a method to support diagnosis and treatment of arterial lesions, according to an abundant that includes computed tomography image acquisition;

FIG. 14 is a flowchart of a method to support diagnosis and treatment of arterial lesions, according to an embodiment that includes computed tomography image acquisition;

FIG. 15 is a dataflow diagram of a method to support diagnosis and treatment of arterial lesions, according to an embodiment that includes comparison of lesions from multiple imaging studies;

FIG. 16 is a block diagram of a hardware and operating environment in which different embodiments can be practiced; and

FIG. 17 is a block diagram of the apparatus in which an arterial plaque image change analyzer is implemented.

### DETAILED DESCRIPTION OF THE INVENTION

In the following detailed description, reference is made to the accompanying drawings that form a part hereof, and in which is shown by way of illustration specific embodiments which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

The detailed description is divided into five sections. In the first section, a system level overview is described. In the second section, embodiments of methods are described. In the third section, a hardware and the operating environment in conjunction with which embodiments may be practiced are described. In the fourth section, particular implementations are described. Finally, in the fifth section, a conclusion of the detailed description is provided.

### System Level Overview

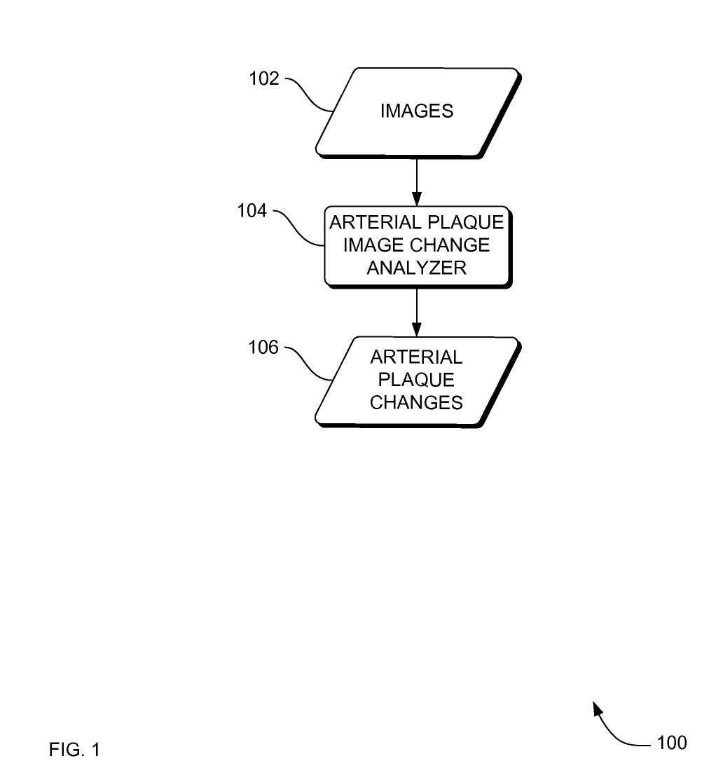

FIG. 1 is a block diagram of an overview of a system 100 to support diagnosis and treatment of arterial lesions, according to an embodiment. System 100 solves the need in the art to track changes in coronary arterial plaque lesions over time.

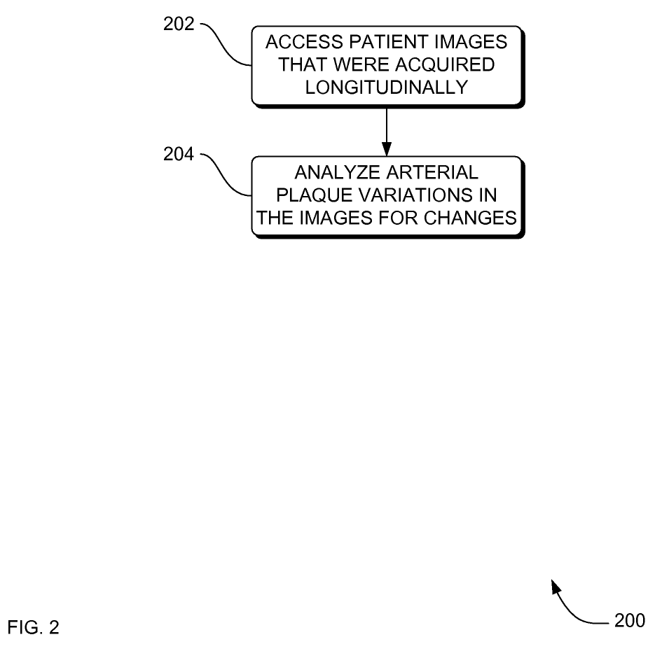

System 100 includes a set of images 102 that are received by an arterial plaque image change analyzer 104. The arterial plaque image change analyzer 104 is operable to identify one or more arterial plaque change(s) 106 in the images 102. Various embodiments of processes that the arterial plaque image change analyzer 104 is operable to perform are described below in method FIGS. 2-15.

While the system 100 is not limited to any particular set of images 102, arterial plaque image change analyzer 104, arterial plaque change 106, for sake of clarity a simplified set of images 102, arterial plaque image change analyzer 104, arterial plaque change 106 are described.

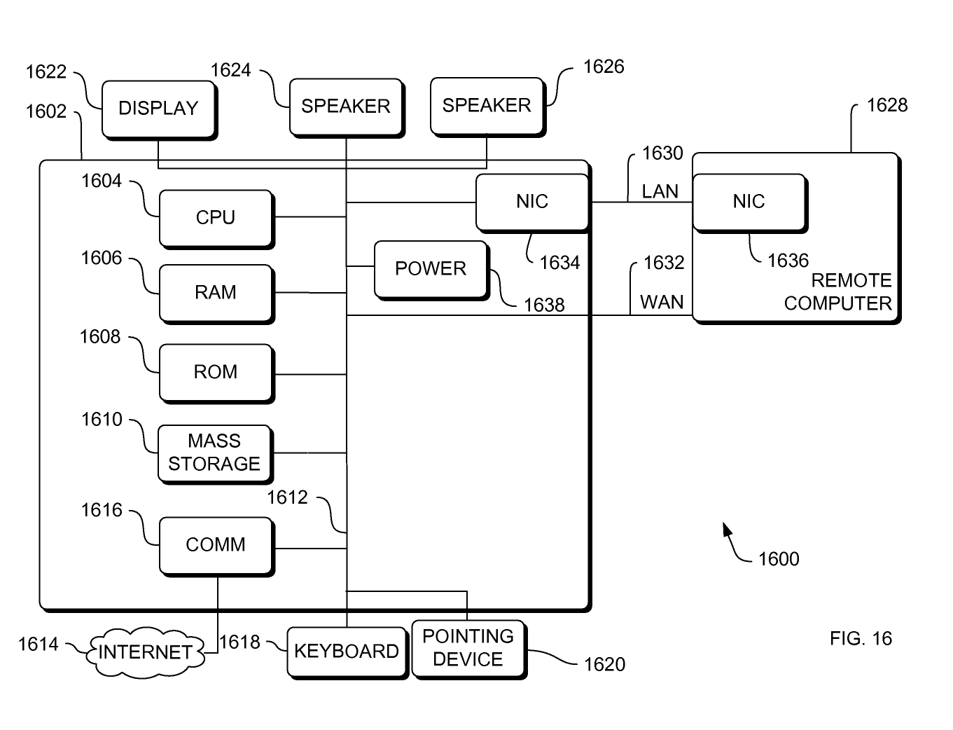

The system level overview of the operation of an embodiment is described above in this section of the detailed description. Some embodiments operate in a multi-processing, multi-threaded operating environment on a computer, such as computer 1602 in FIG. 16.

### Method Embodiments

In the previous section, a system level overview of the operation of an embodiment is described. In this section, the particular methods of such an embodiment are described by reference to a series of flowcharts. Describing the methods by reference to a flowchart enables one skilled in the art to develop such programs, firmware, or hardware, including such instructions to carry out the methods on suitable computers, executing the instructions from computer-readable media. Similarly, the methods performed by the server computer programs, firmware, or hardware are also composed of computer-executable instructions. Methods 200-1500 are performed by a program executing on, or performed by firmware or hardware that is a part of, a computer, such as computer 1602 in FIG. 16.

FIG. 2 is a flowchart of a method 200 to support diagnosis and treatment of arterial lesions, according to an embodiment. Method 200 solves the need in the art to track changes in coronary arterial plaque lesions over time.

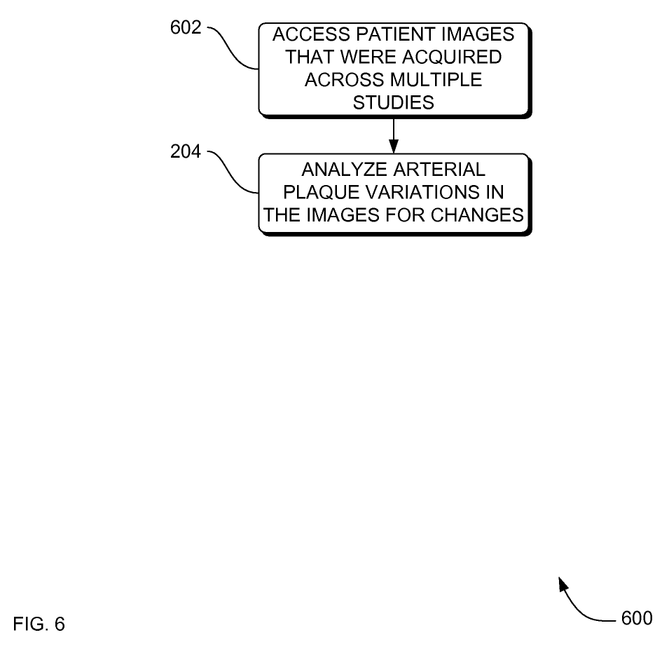

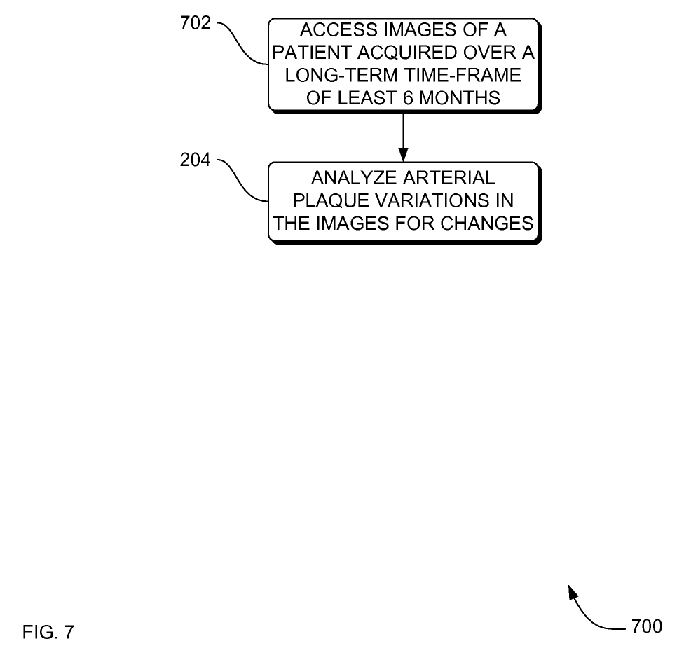

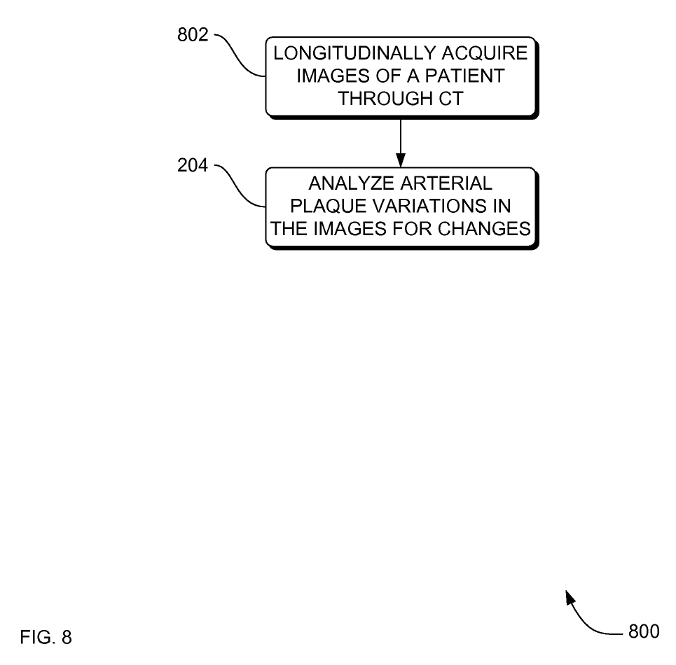

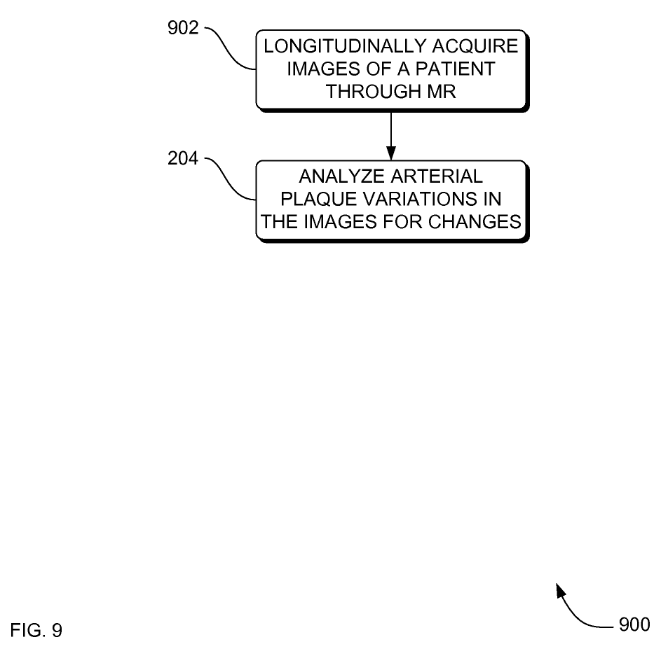

Method 200 includes accessing 202 longitudinally, a plurality of images of a patient. Various embodiments of acquiring 102 are described below in FIGS. 8-9. Various embodiments of the longitudinal aspect are described below in FIGS. 6-7.

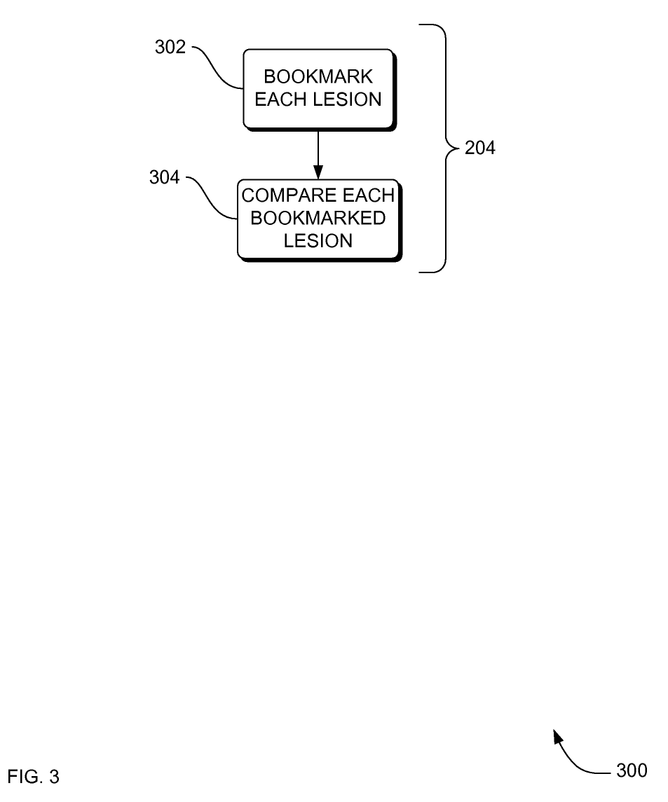

Method 200 also includes analyzing 204 arterial plaque variations in the plurality of images for changes. In some embodiments the changes are selected from a group of changes that include in their limited to one or more changes in size of the arterial plaque and, one or more changes in composition of the arterial plaque, and one or more changes in location of the arterial plaque. Various embodiments of the analyzing 204 are described below in the FIGS. 3-5.

FIG. 3 is a flowchart of a method 300 of analyzing the plurality of images for changes in arterial plaque. Method 300 is one embodiment of analyzing 204 arterial plaque variations in a plurality of images for changes, in FIG. 2 above.

In some embodiments method 300 includes bookmarking 302 each lesion. In some embodiments bookmarking 302 is performed at the manual and direction of a user through a graphical user interface.

In some embodiments FIG. 3 includes longitudinally comparing 304 each bookmarked lesion. In some embodiments the comparing through a four is performed with registration and in some embodiments, the comparing a 304 is performed with no registration.

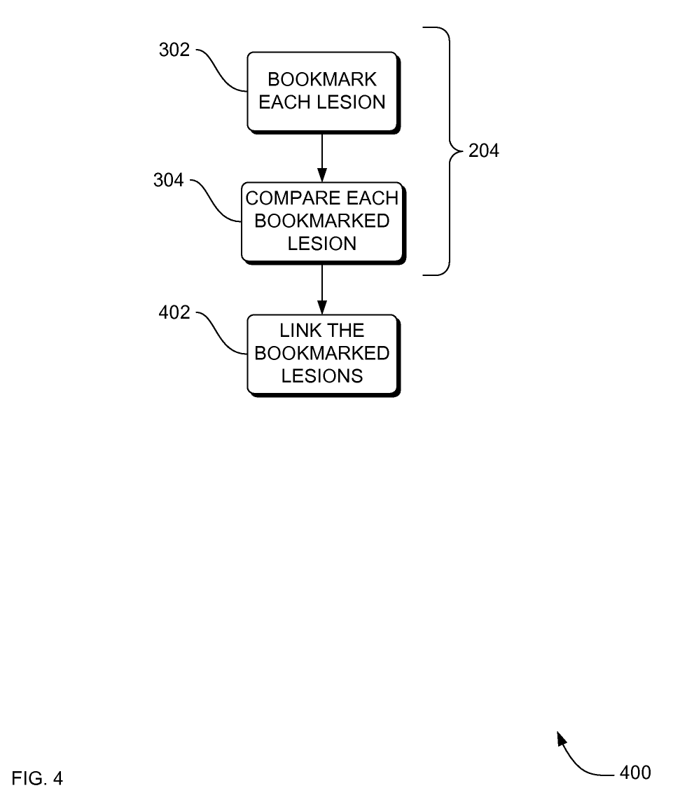

FIG. 4 is a flowchart of a method 400 analyzing the plurality of images for changes in arterial plaque. Method 400 is one embodiment of analyzing 204 arterial plaque variations in a plurality of images for changes, as in FIG. 2 above.

Method 400 includes bookmarking 302 and longitudinally comparing 304. Method 400 also include linking 402 the bookmarked lesions on a vessel by vessel basis. Some embodiments of the linking 402 are performed in reference to a standard reference such as an atlas.

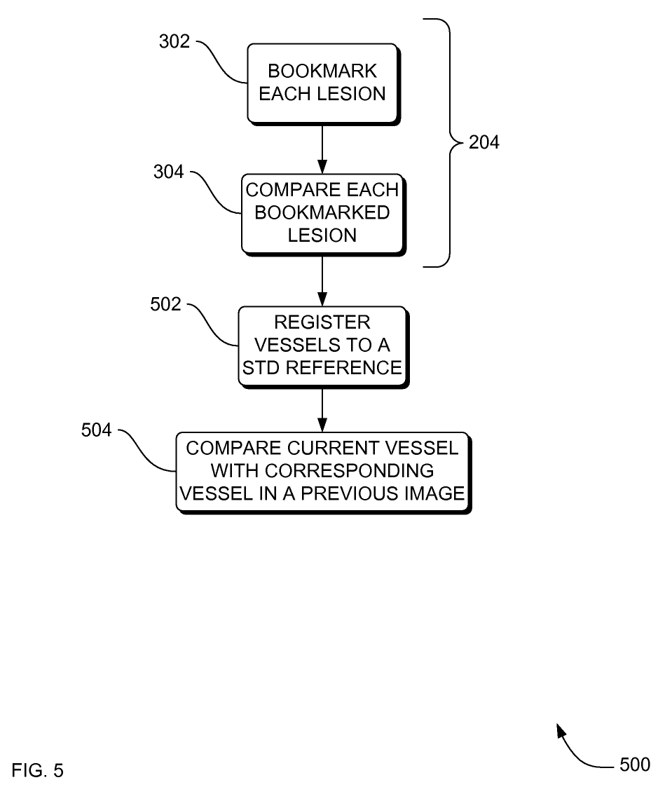

FIG. 5 is a flowchart of a method 500 analyzing the plurality of images for changes in arterial plaque. Method 500 is one embodiment of analyzing 204 arterial plaque variations in a plurality of images for changes, as in FIG. 2 above.

Method 500 includes bookmarking 302 and longitudinally comparing 304. Some embodiments of method 500 include registering 502 vessels to a common reference or a standard reference, such as an atlas. Some embodiments of method 500 also includes comparing 504 a current vessel with the corresponding vessel in a previous image. More specifically, in the comparing 504, a plurality of corresponding vessels, in the plurality of images are compared.

FIG. 6 is a flowchart of a method 600 to support diagnosis and treatment of arterial lesions, according to an embodiment of longitudinal image acquisition. Method 600 solves the need in the art to track changes in coronary arterial plaque lesions over time. Method 600 includes accessing 602 a plurality of images of a patient that were acquired across multiple studies. Method 600 also includes analyzing 204 arterial plaque variations in the plurality of images for changes as described in FIG. 2 above.

FIG. 7 is a flowchart of a method 700 to support diagnosis and treatment of arterial lesions, according to an embodiment of longitudinal image acquisition. Method 700 solves the need in the art to track changes in coronary arterial plaque lesions over time. Method 700 includes accessing 702 images of a patient wherein the images were acquired over a long-term timeframe. One example of a long-term timeframe is at least six months.

Method 700 also includes analyzing 204 arterial plaque variations in the plurality of images for changes as described in FIG. 2 above.

FIG. 8 is a flowchart of a method 800 to support diagnosis and treatment of arterial lesions, according to an embodiment that includes computed tomography image acquisition. Method 800 includes acquiring 802 the images through computed tomography. Method 800 also includes analyzing 204 arterial plaque variations in the plurality of images for changes as described in FIG. 2 above.

FIG. 9 is a flowchart of a method to support diagnosis and treatment of arterial lesions, according to an embodiment that includes magnetic resonance image acquisition. Method 900 includes acquiring 902 the images through magnetic resonance imaging. Method 800 also includes analyzing tool for arterial plaque variations in the plurality of images for changes as described in FIG. 2 above.

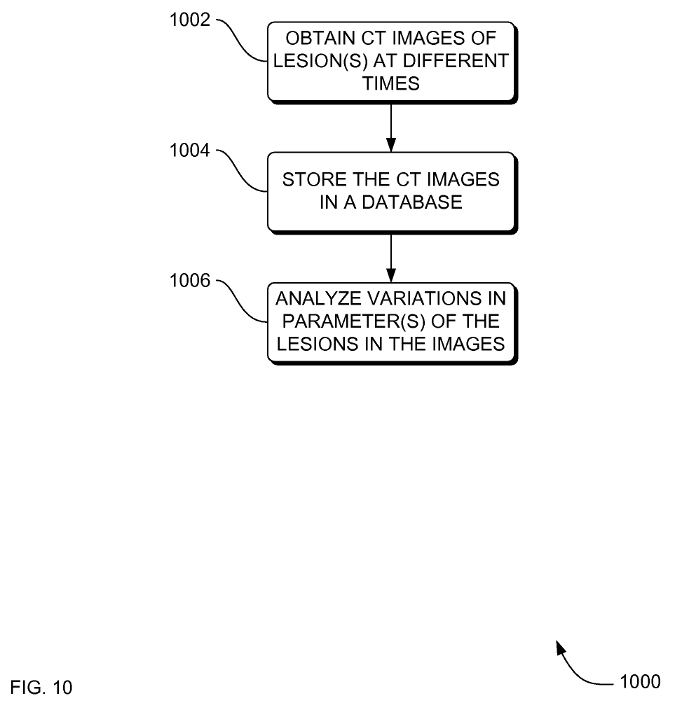

FIG. 10 is a flowchart of a method 1000 to support diagnosis and treatment of arterial lesions, according to an embodiment that includes computed tomography image acquisition. Method 1000 includes accessing and/or obtaining 1002 a plurality of sets of computed-tomography (CT) images. The CT images include a representation of at least one arterial plaque lesion. Each set of the CT images are acquired at a different time. Various embodiments of the accessing/obtaining 1002 are described above in FIGS. 8-9.

Method 1000 also includes storing 1004 the CT images in a database. Method 1000 also includes analyzing 1006 arterial plaque variations in the sets of SCT images. For changes in one or more parameters (attributes) in the arterial plaque lesions. Various embodiments of the analyzing 1006 are described above in FIGS. 3-5 and FIGS. 11-12 and below.

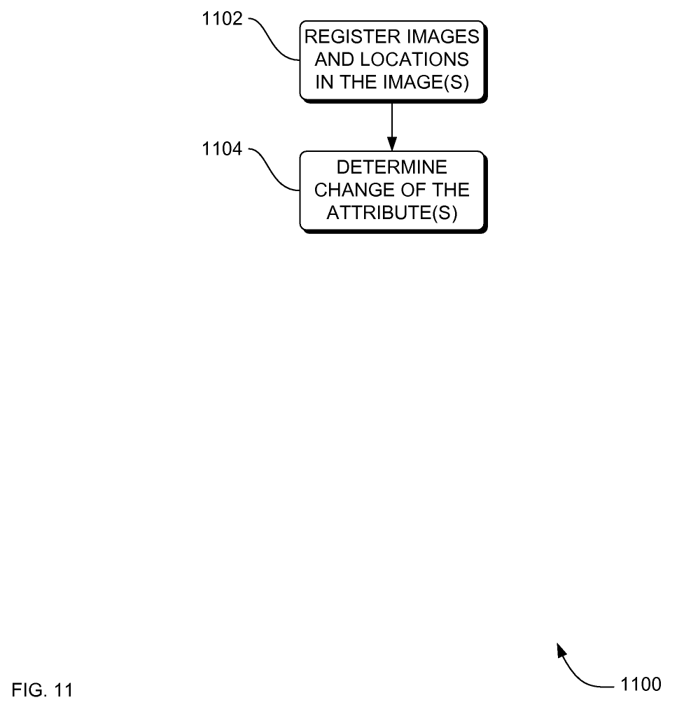

FIG. 11 is a flowchart of a method 1100 to support diagnosis and treatment of arterial lesions, according to an embodiment. Method 1100 includes registering 1102 images and locations in the images. In some embodiments method 1100 also includes determining 1104 changes in each of the one or more parameters between different times in each of the one or more arterial plaque lesions in the sets of CT images.

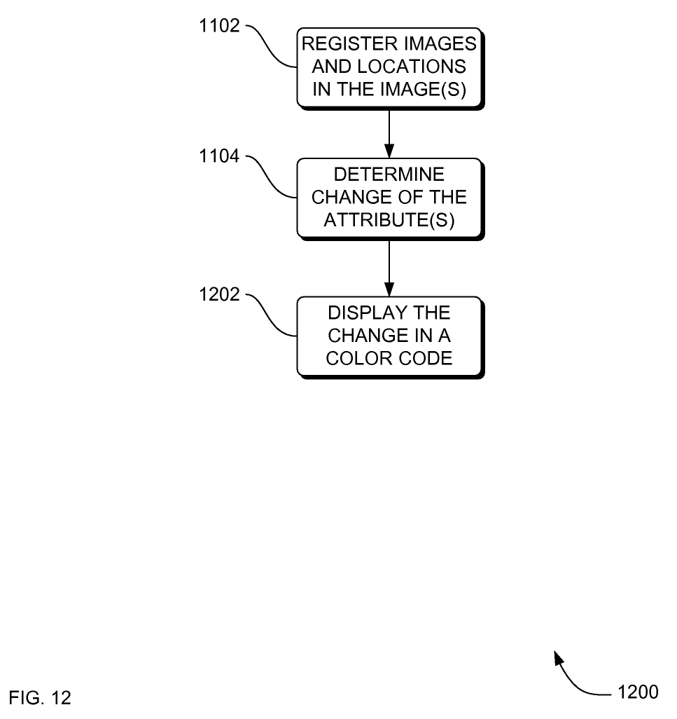

FIG. 12 is a flowchart of a method 1200 to support diagnosis and treatment of arterial lesions, according to an embodiment that provides a visual cue of changes. Method 1200 includes registering 1102 and determining 1104. Method 1200 also includes displaying 1202 changes. In some embodiments the changes are displayed in a color code that represents positive and negative change of each parameter or attribute. Method 1200 is a method to graphically and interactively follow the temporal changes in plaque deposits in a patient and evaluate the effects of drugs on these deposits.

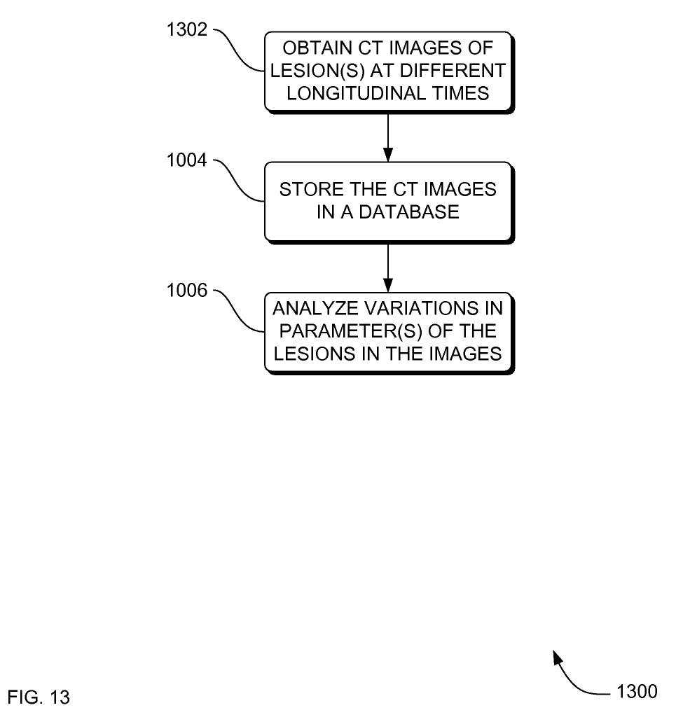

FIG. 13 is a flowchart of a method 1300 to support diagnosis and treatment of arterial lesions, according to an embodiment that includes computed tomography image acquisition. Method 1300 includes accessing and/or obtaining 1302 a plurality of sets of computed-tomography (CT) images. The CT images include a representation of at least one arterial plaque lesion. Each set of the CT images are acquired at a longitudinal different time. Various embodiments of accessing/obtaining 1002 are described above in FIGS. 8-9.

Method 1300 also includes storing 1004 the CT images in a database. Method 1300 also includes analyzing 1006 arterial plaque variations in the sets of SCT images, for changes in one or more parameters (attributes) in the arterial plaque lesions. Various embodiments of the analyzing 1006 are described above in FIGS. 3-5 and FIGS. 11-12 and above.

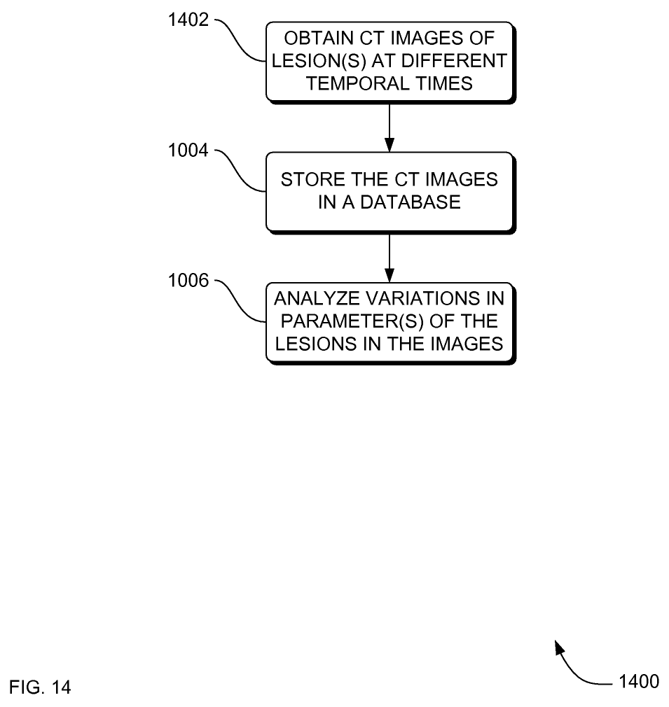

FIG. 14 is a flowchart of a method 1400 to support diagnosis and treatment of arterial lesions, according to an embodiment that includes computed tomography image acquisition. Method 1400 includes accessing and/or obtaining 1402 a plurality of sets of computed-tomography (CT) images. The CT images include a representation of at least one arterial plaque lesion. Each set of the CT images are acquired at a different temporal time. Various embodiments of accessing/obtaining 1402 are described above in FIGS. 8-9.

Method 1400 also includes storing 1004 the CT images in a database. Method 1400 also includes analyzing 1006 arterial plaque variations in the sets of SCT images. For changes in one or more parameters (attributes) in the arterial plaque lesions. Various embodiments of the analyzing 1006 are described above in FIGS. 3-5 and FIGS. 11-12 and above.

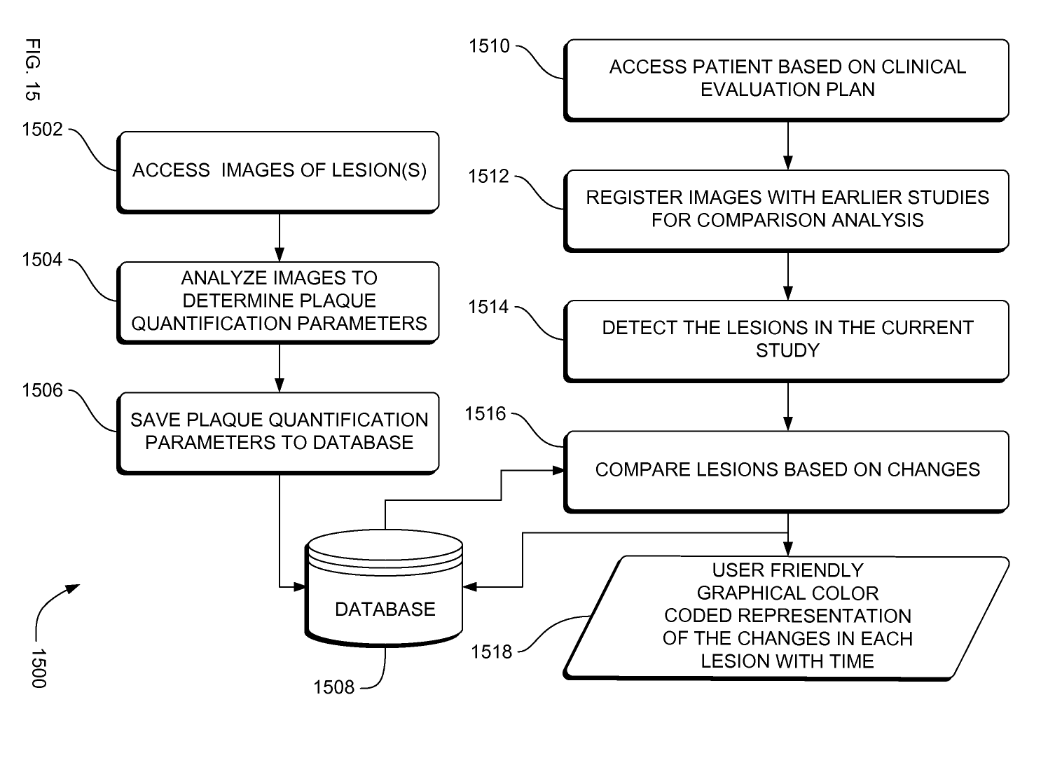

FIG. 15 is a dataflow diagram of a method 1500 to support diagnosis and treatment of arterial lesions, according to an embodiment that includes comparison of lesions from multiple imaging studies. In general, method 1500 requires that CT images of a patent are accessed/obtained and plaque lesion information is collected and stored in a database, the patient comes back for a follow-up CT study, new information about plaque lesions of the patient is collected and plaque lesions of the patient are analyzed for changes in size, composition, characteristics and location, and the historical data is reported.

A particular method 1500 of study of a patient includes accessing 1502 images of one of more lesions of a patient and analyzing 1504 the images to determine plaque quantification parameters. Thereafter, the plaque quantification parameters are saved 1506 to a database 1508.

Some embodiments of the particular method 1500 of study of a patient also include accessing 1510 the scanned patient images again. The scan is based on a clinical evaluation plan and in some cases, can be very similar to accessing 1502 images of one of more lesions of the patient.

Some embodiments of the particular method 1500 of study of a patient also include registering 1512 the images with one or more earlier study(s) for comparison analysis.

Some embodiments of the particular method 1500 of study of a patient also includes detecting 1514 the lesions in the current study. In some embodiments, the detecting 1514 is performed using, based on, or in reference to lesion information from the earlier study(s).

Some embodiments of the particular method 1500 of study of a patient also includes comparing 1516 lesions based on changes in size, location, density, volume, composition, topology, remodeling etc. and saving current results for next study and building the historical profile (not shown).

Some embodiments of the particular method 1500 of study of a patient also include generating or presenting 1518 a user friendly graphical color coded representation of the changes in each lesion with time.

Method 1500 graphically and interactively tracks and follows temporal changes in plaque deposits in the patient and allows healthcare practitioners to evaluate the effects of drugs on these deposits.

In some embodiments, methods 200-1500 are implemented as a computer data signal embodied in a carrier wave, that represents a sequence of instructions which, when executed by a processor, such as processor 1604 in FIG. 16, cause the processor to perform the respective method. In other embodiments, methods 200-1500 are implemented as a computer-accessible medium having executable instructions capable of directing a processor, such as processor 1604 in FIG. 16, to perform the respective method. In varying embodiments, the medium is a magnetic medium, an electronic medium, or an optical medium.

### Hardware and Operating Environment

FIG. 16 is a block diagram of a hardware and operating environment 1600 in which different embodiments can be practiced. The description of FIG. 16 provides an overview of computer hardware and a suitable computing environment in conjunction with which some embodiments can be implemented. Embodiments are described in terms of a computer executing computer-executable instructions. However, some embodiments can be implemented entirely in computer hardware in which the computer-executable instructions are implemented in read-only memory. Some embodiments can also be implemented in client/server computing environments where remote devices that perform tasks are linked through a communications network. Program modules can be located in both local and remote memory storage devices in a distributed computing environment.

Computer 1602 includes a processor 1604, commercially available from Intel, Motorola, Cyrix and others. Computer 1602 also includes random-access memory (RAM) 1606, read-only memory (ROM) 1608, and one or more mass storage devices 1610, and a system bus 1612, that operatively couples various system components to the processing unit 1604. The memory 1606, 1608, and mass storage devices, 1610, are types of computer-accessible media. Mass storage devices 1610 are more specifically types of nonvolatile computer-accessible media and can include one or more hard disk drives, floppy disk drives, optical disk drives, and tape cartridge drives. The processor 1604 executes computer programs stored on the computer-accessible media.

Computer 1602 can be communicatively connected to the Internet 1614 via a communication device 1616. Internet 1614 connectivity is well known within the art. In one embodiment, a communication device 1616 is a modem that responds to communication drivers to connect to the Internet via what is known in the art as a “dial-up connection.” In another embodiment, a communication device 1616 is an Ethernet® or similar hardware network card connected to a local-area network (LAN) that itself is connected to the Internet via what is known in the art as a “direct connection” (e.g., T1 line, etc.).

A user enters commands and information into the computer 1602 through input devices such as a keyboard 1618 or a pointing device 1620. The keyboard 1618 permits entry of textual information into computer 1602, as known within the art, and embodiments are not limited to any particular type of keyboard. Pointing device 1620 permits the control of the screen pointer provided by a graphical user interface (GUI) of operating systems such as versions of Microsoft Windows®. Embodiments are not limited to any particular pointing device 1620. Such pointing devices include mice, touch pads, trackballs, remote controls and point sticks. Other input devices (not shown) can include a microphone, joystick, game pad, satellite dish, scanner, or the like.

In some embodiments, computer 1602 is operatively coupled to a display device 1622. Display device 1622 is connected to the system bus 1612. Display device 1622 permits the display of information, including computer, video and other information, for viewing by a user of the computer. Embodiments are not limited to any particular display device 1622. Such display devices include cathode ray tube (CRT) displays (monitors), as well as flat panel displays such as liquid crystal displays (LCD's). In addition to a monitor, computers typically include other peripheral input/output devices such as printers (not shown). Speakers 1624 and 1626 provide audio output of signals. Speakers 1624 and 1626 are also connected to the system bus 1612.

Computer 1602 also includes an operating system (not shown) that is stored on the computer-accessible media RAM 1606, ROM 1608, and mass storage device 1610, and is executed by the processor 1604. Examples of operating systems include Microsoft Windows®, Apple MacOS®, Linux®, UNIX®. Examples are not limited to any particular operating system, however, and the construction and use of such operating systems are well known within the art.

Embodiments of computer 1602 are not limited to any type of computer 1602. In varying embodiments, computer 1602 comprises a PC-compatible computer, a MacOS®-compatible computer, a Linux®-compatible computer, or a UNIX®-compatible computer. The construction and operation of such computers are well known within the art.

Computer 1602 can be operated using at least one operating system to provide a graphical user interface (GUI) including a user-controllable pointer. Computer 1602 can have at least one web browser application program executing within at least one operating system, to permit users of computer 1602 to access an intranet, extranet or Internet world-wide-web pages as addressed by Universal Resource Locator (URL) addresses. Examples of browser application programs include Netscape Navigator® and Microsoft Internet Explore®.

The computer 1602 can operate in a networked environment using logical connections to one or more remote computers, such as remote computer 1628. These logical connections are achieved by a communication device coupled to, or a part of, the computer 1602. Embodiments are not limited to a particular type of communications device. The remote computer 1628 can be another computer, a server, a router, a network PC, a client, a peer device or other common network node. The logical connections depicted in FIG. 16 include a local-area network (LAN) 1630 and a wide-area network (WAN) 1632. Such networking environments are commonplace in offices, enterprise-wide computer networks, intranets, extranets and the Internet.

When used in a LAN-networking environment, the computer 1602 and remote computer 1628 are connected to the local network 1630 through network interfaces or adapters 1634, which is one type of communications device 1616. Remote computer 1628 also includes a network device 1636. When used in a conventional WAN-networking environment, the computer 1602 and remote computer 1628 communicate with a WAN 1632 through modems (not shown). The modem, which can be internal or external, is connected to the system bus 1612. In a networked environment, program modules depicted relative to the computer 1602, or portions thereof, can be stored in the remote computer 1628.

Computer 1602 also includes power supply 1638. Each power supply can be a battery.

### Implementation

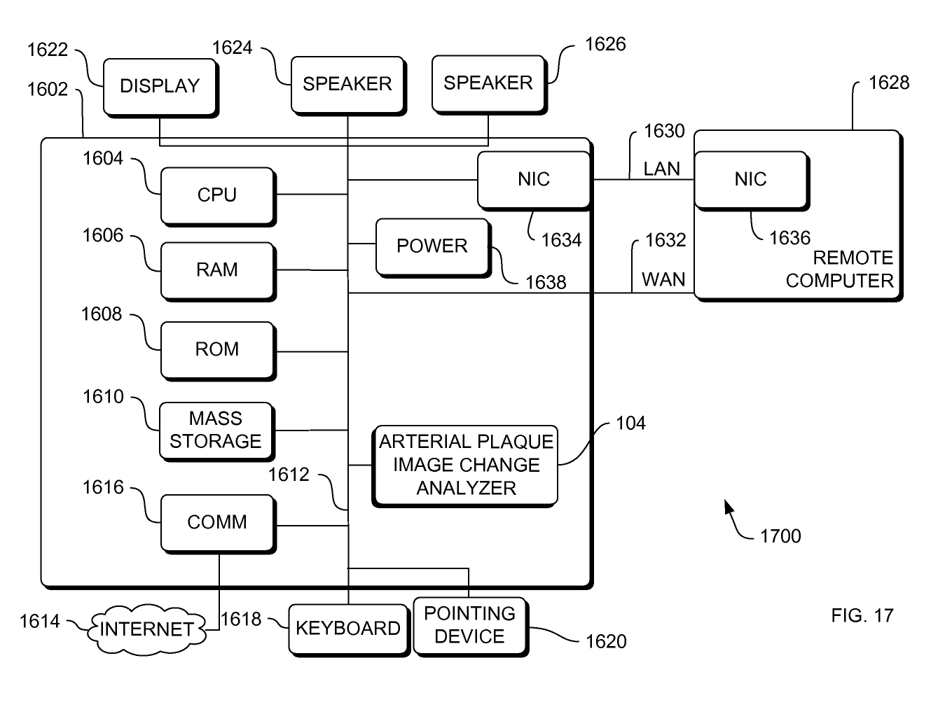

Referring to FIG. 17, a particular implementation 1700 is described in conjunction with the system overview in FIG. 16 and the methods described in conjunction with FIGS. 2-15.

Apparatus 1700 solves the need in the art to track changes in coronary arterial plaque lesions over time.

Apparatus 1700 includes an arterial plaque image change analyzer 104 as in FIG. 1 above that is operable to identify at least one arterial plaque change in the images 102.

Apparatus 104 component and the actions of FIG. 2-15 can be embodied as computer hardware circuitry or as a computer-readable program, or a combination of both. In other embodiments, system 100, methods 200-1500 and apparatus 1600 are implemented in an application service provider (ASP) system.

More specifically, in the computer-readable program embodiment, the programs can be structured in an object-orientation using an object-oriented language such as Java, Smalltalk or C++, and the programs can be structured in a procedural-orientation using a procedural language such as COBOL or C. The software components communicate in any of a number of means that are well-known to those skilled in the art, such as application program interfaces (API) or interprocess communication techniques such as remote procedure call (RPC), common object request broker architecture (CORBA), Component Object Model (COM), Distributed Component Object Model (DCOM), Distributed System Object Model (DSOM) and Remote Method Invocation (RMI). The components execute on as few as one computer as in computer 1602 in FIG. 16, or on at least as many computers as there are components.

### Conclusion

An arterial plaque image change analyzer is described. A technical effect of the arterial plaque image change analyzer is to identify changes in cardiac arterial lesions. Plaque deposits in patients that are termed clinically risky or are being treated, are usually followed up non-invasively with CT scanning. The following method can use the follow-up scans to provide an accurate determination of the changes in plaque deposits in patients over time.

In some embodiments, a plaque longitudinal application includes any of the following: Acquire images of the patient vessels of interest and determine the risk of the deposits. All quantification parameters such as length, volume, composition, location, topology, remodeling, percent of stenosis of the plaque deposit region is detected, measured and stored in a database for the patient. The patient is scanned again for follow-up studies using the same procedure at clinically determined time intervals. The previous information from the database are used to find the same lesions in the vessels. The plaque quantification parameters are measured again for each lesion from the new scan. The new measurements will go into the history of the patient, and are compared against results from each previous scan. Each follow up study provides a time point in the patient temporal disease history. The changes in each lesion since previous study are determined and graphically represented using a color code that reflects the positive and negative changes in each parameter of interest. The method will provide a graphical representation of the change in size, composition, topology, remodeling and location of each plaque deposit of interest. Provide a historical database of each parameter of plaque quantification for analysis. The analysis will use registration techniques to register images and locations in images collected at different times for an accurate determination and quantification of each lesion of interest.

Although specific embodiments have been illustrated and described herein, it will be appreciated by those of ordinary skill in the art that any arrangement which is calculated to achieve the same purpose may be substituted for the specific embodiments shown. This application is intended to cover any adaptations or variations. For example, although described in procedural terms, one of ordinary skill in the art will appreciate that implementations can be made in an object-oriented design environment or any other design environment that provides the required relationships.

In particular, one of skill in the art will readily appreciate that the names of the methods and apparatus are not intended to limit embodiments. Furthermore, additional methods and apparatus can be added to the components, functions can be rearranged among the components, and new components to correspond to future enhancements and physical devices used in embodiments can be introduced without departing from the scope of embodiments. One of skill in the art will readily recognize that embodiments are applicable to future communication devices, different file systems, and new data types.

The terminology used in this application is meant to include all object-oriented, database and communication environments and alternate technologies which provide the same functionality as described herein.

We claim:

1. A computer-accessible medium having executable instructions to support s diagnosis and treatment of arterial lesions, the executable instructions capable of directing a processor to perform:
accessing a plurality of patient images that were acquired longitudinally; and
analyzing arterial plaque variations in the plurality of images for changes selected from a group of changes consisting of at least one change in size, at least one change in composition, at least one change in characteristics and at least one change in location.

2. The computer-accessible medium of claim 1, wherein the executable instructions capable of directing the processor to perform the analyzing further comprise executable instructions capable of directing the processor to perform:
bookmarking each lesion; and
comparing longitudinally each bookmarked lesion.

3. The computer-accessible medium of claim 1, wherein the executable instructions capable of directing the processor to perform the analyzing further comprise executable instructions capable of directing the processor to perform:
linking the bookmarked lesions on a vessel-by-vessel basis.

4. The computer-accessible medium of claim 1, wherein the executable instructions capable of directing the processor to perform the analyzing further comprise executable instructions capable of directing the processor to perform:
registering vessels to a common reference or standard reference; and
comparing the current vessel with the corresponding vessel in a previous image.

5. The computer-accessible medium of claim 1, wherein longitudinally further comprises:
across multiple studies.

6. The computer-accessible medium of claim 1, wherein longitudinally further comprises:
over a long-term time frame.

7. The computer-accessible medium of claim 1, wherein the executable instructions capable of directing the processor to perform the acquiring further comprise executable instructions capable of directing the processor to perform:
acquiring through computed-tomography.

8. The computer-accessible medium of claim 1, wherein the executable instructions capable of directing the processor to perform the acquiring further comprise executable instructions capable of directing the processor to perform:
acquiring through magnetic resonance.

9. A method to support diagnosis and treatment of arterial lesions, the method comprising:
obtaining a plurality of sets of computed-tomography images of at least one arterial plaque lesion, wherein each set of computed-tomography images are acquired at a different time;
storing the computed-tomography images in a database; and
analyzing arterial plaque variations in the sets of computed-tomography images for changes in at least one parameter.

10. The method of claim 9, the analyzing further comprising:
bookmarking each lesion; and
comparing longitudinally each bookmarked lesion.

11. The method of claim 9, the analyzing further comprising:
registering images and locations in the images; and
determining changes in each of the at least one parameter between the different times in each of the at least one arterial plaque lesion in the sets of computed-tomography images.

12. The method of claim 11, wherein the method further comprises:
displaying the changes in a color code that represents positive and negative change of each parameter.

13. The method of claim 9, wherein the at least one parameter further comprises:
size, composition, characteristics and/or location.

14. The method of claim 9, wherein the different time further comprises:
different longitudinal time.

15. The method of claim 9, wherein the different time further comprises:
different temporal time.

16. A system comprising:
a processor;
a storage device coupled to the processor;
software apparatus operative on the processor to:
detect changes in cardiovascular arterial lesions based on a plurality of studies at a plurality of times; and
generate a graphical color coded representation of the changes in each cardiovascular arterial lesion with the plurality of times.

17. The system of claim 16, wherein the software apparatus is further operable to:
compare cardiovascular arterial lesions based on changes in size, location, density, volume, composition, topology, and remodeling.

18. The system of claim 16, wherein the software apparatus is further operable to:
access images of one of more cardiovascular arterial lesions of a patient; and
analyze the images to determine plaque quantification parameters.

19. The system of claim 18, wherein the software apparatus is further operable to:
save plaque quantification parameters to a database.

20. The system of claim 18, wherein the software apparatus is further operable to:
register the images with image from one or more earlier studyies for comparison analysis.
